(19) United States
(12) Patent Application Publication
(10) Pub. No.: US 2025/0177969 A1
(43) Pub. Date: Jun. 5, 2025

(54) LATERAL FLOW ASSAY FOR QUANTITATIVE AND ULTRASENSITIVE DETECTION OF ANALYTE

(71) Applicant: Massachusetts Institute of Technology, Cambridge, MA (US)

(72) Inventors: Timothy M. Swager, Newton, MA (US); Shaoxiong Luo, Cambridge, MA (US); Weize Yuan, Cambridge, MA (US); Jie Li, Waltham, MA (US); Mate J. Bezdek, Boston, MA (US)

(73) Assignee: Massachusetts Institute of Technology, Cambridge, MA (US)

(21) Appl. No.: 18/837,547

(22) PCT Filed: Feb. 9, 2023
(86) PCT No.: PCT/US2023/062268

§ 371 Date: Aug. 9, 2024

Related U.S. Application Data
(60) Provisional application No. 63/339,728, filed on May 9, 2022.
(60) Provisional application No. 63/308,630, filed on Feb. 10, 2022.

(51) Int. Cl. B01L 3/00; G01N 33/543

(52) U.S. Cl. CPC B01L 3/5023; G01N 33/54388; B01L 2300/023; B01L 2300/0636; B01L 2300/0663; B01L 2300/0825

(57) ABSTRACT

A lateral flow device, system and method can be used to detect an analyte by monitoring a change in an electronic property.

### CLAIM OF PRIORITY

This application claims priority to U.S. Provisional Patent Application No. 63/308,630, filed Feb. 10, 2022 and U.S. Provisional Patent Application No. 63/339,728, filed May 9, 2022, each of which is incorporated by reference in its entirety.

### FIELD OF THE INVENTION

The invention relates to lateral flow assay systems and methods.

### BACKGROUND

Many biosensing methods rely on signals produced by enzyme-catalyzed reactions and efficient methods to detect and record this activity. Other chemical sensing methods rely on other chemical interactions and reactions to provide responses.

### SUMMARY

In general, a new type of lateral flow device can detect an analyte by monitoring a change in an electronic property.

In one aspect, a device for detecting an analyte can include a porous substrate and a sensing region including a semiconducting material on a surface of the porous substrate capable of transporting a sample through capillary action. The sensing region can have an electronic property that changes in response to the analyte.

In another aspect, a system for detecting an analyte can include a device as described herein, and a reader configured to measure the electronic property of the sensing region. For example, the reader can include a wireless reader such as a smartphone or any circuit capable of measuring a change in resistance.

In another aspect, a method of manufacturing a device can include depositing a colloidal dispersion on a surface of a substrate to form a semiconducting material. In certain embodiments, the substrate can be a porous substrate. In certain embodiments, this method can be used to create coatings from materials that are nominally insoluble materials, but small fluidized particles can be captured at a liquid-liquid interface of a colloidal dispersion and then used to create conformal coatings on surfaces. In certain circumstances, the method can include reductive functionalization of the semiconducting material to create a film that is in a state optimal for analyte detection by oxidative doping. In some circumstances, a semiconducting material can be deposited by polymerization on a substrate.

In another aspect, a method of detecting an analyte can include exposing a device as described herein, and detecting the analyte in the sample. In certain circumstances, detecting the analyte in the sample can be quantitative.

In certain circumstances, the semiconducting material can include a semiconducting polymer film, a carbon nanotube, or an MXene film, such as a metal carbide or nitride, on the surface of the porous substrate. For example, the semiconducting polymer film can include a polypyrrole, a polyaniline, a polythiophene, a polyacetylene, a polyphenylene, a polyarylene, a polyarylene vinylene, or a polyphenylene vinylene, or combinations thereof. The polymers deposited on a surface can be initially prepared in a form that is conducive for the detection of an analyte. In some embodiments, the polymers are in a more conducting form caused by oxidative doping and an analyte triggered event reduces the conductivity by pinning or eliminating charge carriers. In some embodiments, the polymers are in a less conductive state and an analyte triggered event injects new charge carriers or releases pinned carriers and increases the conductivity. In certain circumstances, the semiconducting material can be initially in a lower conductive state than that after analyte detection. In other circumstances, the semiconducting material can be initially in a higher conductive state than that after analyte detection.

In certain circumstances, the analyte detection results in a change in conductivity of at least 10%, at least 20%, at least 30%, at least 40%, at least 50%, at least 60%, at least 70%, at least 80%, at least 90%, at least 95%, or about 100%.

In certain circumstances, the semiconducting material can include a recognition moiety configured to respond to the analyte. The recognition moiety can include an enzyme, protein, synthetic receptor, antibody, nano-body, nucleic acid, molecular catalyst, metal binding site, a Lewis base, a Lewis acid, or combinations thereof. In certain circumstances, a recognition moiety can be attached to a material upon which the semiconducting material is deposited.

In certain circumstances, the device can include a catalyst adjacent to the semiconducting material. The catalyst can be immbolized adjacent to the semiconducting material by action of an analyte.

In certain circumstances, the device can include a redox catalyst initially in an analyte solution capable of reacting with the semiconducting material.

In certain circumstances, the sensing region can include an enzyme. The sensing region can include adjacent functional groups. For example, enzymes can be captured upstream of the semiconducting material. In another example, a flow regulation element can be downstream of the semiconductor material.

In certain circumstances, the analyte can include a bacterium, a protein, a virus, a nucleic acid, a cell, a biomolecule, a drug, a toxin, a biomarker, a reactive biomarker, an enzyme substrate, a carbohydrate, a metal (for example, a toxic metal), a toxin, a toxic molecule, a metal ion, a heavy metal (for example, mercury or lead), an ion, an inorganic ion, an organic molecule, a highly fluorinated molecule, an oxidant, a Brønsted acid or a base, or combinations thereof.

In certain circumstances, the analyte can be present in a less than a nanogram/Liter concentration, a less than a 100 picogram/Liter concentration, or a less than a 10 picogram/Liter concentration.

In certain circumstances, the analyte can be capable of binding to or reacting with an enzyme or capable of binding to two or more recognition moieties at the same time.

In certain circumstances, the analyte can participate or initiate one or more reactions that result in oxidation or reduction of the semiconducting materials.

In certain circumstances, the porous substrate can include a control region adjacent to the sensing region.

In certain circumstances, the porous substrate can include a sampling region, the sampling region being adjacent to the control region or the sensing region.

In certain circumstances, a control signal can be a change in electrical resistance.

In certain circumstances, the device can include a redox catalyst that can enhance or accelerate the change in the resistive state of the semiconductor with an analyte trigger. The redox catalyst can enhance or accelerate the change in the resistive state of the semiconductor with an analyte trigger.

In certain circumstances, the electronic property can be resistivity.

In certain circumstances, the device can include a radio frequency identification tag electrically connected to the sensing region. For example, the radio frequency identification tag can include an integrated circuit in parallel with the sensing region. In other embodiments, the radio frequency identification tag can include an integrated circuit in series with the sensing region.

Other aspects, embodiments, and features will be apparent from the following description, the drawings, and the claims.

### BRIEF DESCRIPTION OF DRAWINGS

FIG. 1A depicts a schematic showing a device for detecting an analyte.

FIG. 1B depicts a schematic showing a system for detecting an analyte.

FIGS. 2A-2B depict preparation of carboxylate-functionalized pPy core-shell particles. FIG. 2A is a schematic of the preparation of carboxylate-functionalized pPy core-shell particles. FIG. 2B is an illustration of the conjugation and reduction of pPy core shell particles.

FIGS. 3A-3B depict a schematic of the preparation of pPy core-shell particles and the bursting process. FIG. 3A depicts the preparation of pPy core-shell particles. FIG. 3B depicts the automatic bursting process of carboxylate-functionalized pPy core-shell particles on a glass surface.

FIGS. 4A-4B depict optical images of pPy core-shell particles and pPy core-shell particles after carboxylate-functionalization. FIG. 4A shows an optical image of pPy core-shell particles. FIG. 4B shows an optical image of pPy core-shell particles after carboxylate-functionalization.

FIGS. 5A-5E depict SEM images of pPy core-shell particles with different volume ratio of 3-mercaptopropionic acid. IG. 5A shows no post-functionalizations. FIGS. 5B-5E show the volume ratio of pyrrole: 3-mercaptopropionic acid equals to 5:1, 5:2, 5:3 and 5:4, respectively.

FIGS. 6A-6C depict an illustration of pPy particle bioconjugation. FIG. 6A shows bioconjugation of streptavidin to the carboxylate functionalized pPy particles. FIG. 6B shows a fluorescent microscope image of pPy particles after bioconjugation of streptavidin and addition of AFDye 594 biotin for 2 h. Red fluorescence indicates the success of bioconjugation on the particles. Scale bar=50 μm. FIG. 6C shows a schematic illustration of the bioconjugation of biotinylated glucose oxidase to the pPy core-shell particles. Only one of the four potential biotin-streptavidin sites are shown as occupied for clarity.

FIGS. 7A-7B depict fluorescent microscope images of pPy core-shell particles after NHS activation. FIG. 7A shows a fluorescent microscope image. FIG. 7B shows a bright-field microscope image. Scale bars=50 μm.

FIG. 8 depicts a schematic illustration of the bioconjugation of pyruvate oxidase to the pPy core-shell particles. Only one of the four potential biotin-streptavidin sites is shown to be occupied for clarity.

FIGS. 9A-9C depict a preparation of a Lateral Flow Device. FIG. 9A shows a schematic illustration of the conformal coating process. FIG. 9B shows a demonstration of the composition of the lateral flow device. FIG. 9C shows an optical image of the lateral flow device.

FIGS. 10A-10C depict SEM images of the nitrocellulose membrane. FIG. 10A shows an SEM image of the nitrocellulose membrane before coating of pPy core-shell particles. FIG. 10B shows an SEM image of the nitrocellulose membrane after coating of bio-conjugated pPy core-shell particles. The white circle on the image signifies the pores on the coated nitrocellulose membrane. FIG. 10C shows an SEM image of the nitrocellulose membrane after coating of bio-conjugated pPy core-shell particles at higher magnification.

FIG. 11 depicts a schematic illustration of the conductivity increase of pPy film conjugated with glucose oxidase or pyruvate oxidase on the LFA when samples containing glucose and sodium pyruvate were added. The doping of pPy film was induced by the production of H2O2 by glucose oxidase or pyruvate oxidase.

FIGS. 12A-12E depict conversion of a commercial NFC tag to a p-CARD for the wireless detection of enzyme response. FIG. 12A shows incorporation of the lateral flow device's test line in parallel with the NFC integrated circuit (IC) by wires. FIG. 12B shows the circuit design of the p-CARD. FIG. 12C shows resonance-frequency traces for the p-CARD with addition of different concentrations of glucose solutions and 0.16 wt % of catalyst. FIG. 12D shows correlation of magnitude of response (ΔGain) at 13.56 MHz and glucose concentration. All error bars are standard deviation calculated from three independent experiments (n=3). FIG. 12E shows a magnitude of response (ΔGain) at 13.56 MHz when adding water or 1 wt % of sodium pyruvate (with 0.16% of catalyst) to the lateral flow device. All error bars are standard deviation calculated from three independent experiments (n=3).

FIG. 13 depicts resonance-frequency traces for p-CARD before and after addition of D.I. water with 0.16 wt % of catalyst.

FIG. 14 depicts resonance-frequency traces for p-CARD (coated by pyruvate oxidase conjugated pPy particles) before and after addition of 1% of sodium pyruvate and 0.16 wt % of catalyst.

FIG. 15 depicts thickness of the pPy film on nitrocellulose membrane measured by profilometer.

FIGS. 16A-16C depict the wireless detection of C-reactive protein (CRP) with the lateral flow devices. FIG. 16A shows an illustration of the composition of the lateral flow device.

FIG. 16B shows a graph of resonance-frequency traces for the p-CARD pre-treated with different concentrations of CRP. 0.2 wt % of glucose and 0.16 wt % of catalyst were finally added to the devices to generate the signals. FIG. 16C shows a graph of correlation of magnitude of response (ΔGain) at 13.56 MHz and concentration of CRP. All error bars are standard deviation calculated from three independent experiments (n=3).

FIG. 17 is a schematic of a lateral flow device.

### DETAILED DESCRIPTION

The formation of a system and method that converts a conventional lateral flow assay (LFA) process to an electronically readable system is described herein. The transducing materials used to detect the analyte are deposited using a colloidal dispersion, printed from solution, or polymerized on the support. In an operational lateral flow device, a sample is wicked through a test strip to give a measurement. The method does not use the conventional optical methods in the detection, but uses changes in the electrical resistivity of a band of material comprising π-conjugated or other semiconductor system that is capable of conductivity changes as a result oxidation or reduction by an enzyme or its product. In an illustrative example poly(pyrrole) is functionalized with oxidase enzymes that produce a local hydrogen peroxide signal in response to analytes. An oxidation (redox) catalyst can be used to efficiently affect an oxidative doping of the polymer in the presence of the hydrogen peroxide. The transduction scheme is general and enzymes that create other oxidizing molecules or alternatively reducing molecules can be localized at the conducting materials to produce the conductivity changes in response to biomolecular recognition events. The ideal paring of the molecule that is responsible for the conductivity change will depend on the type of semiconductor and its functionalization. An n-type semiconductor, or a semiconductor that increases its conductance, when reduced would be better paired with a reducing molecule produced as a result of biomolecular recognition event. A p-type semiconductor that was already oxidatively doped into a conductive state could similarly be triggered to lower its conductivity by a reducing molecule that lowers its doping level. A p-type semiconductor in a low conductivity (low doping level) state can in some embodiments be paired with an oxidizing molecule produced by an enzyme that can cause an increase in its conductivity. Semiconductors can also display changes in conductivity to other molecules or ions and in some cases binding of an anion or cation will change the conductivity.

As an initial demonstration of this LFA concept, the quantitative detection of glucose based on a change in electrical resistance and/or a shift of the resonant frequency of a passive radio-frequency identification (RFID) tag is exemplified below. The system can include an inexpensive reader, which can allow for devices to be read via other wireless methods. For example, wireless methods are of interest for coupling to smartphones for in home diagnostics that can also be optionally monitored centrally. Wireless methods can include methods such as Bluetooth, local wireless networks, or other radio frequencies. In some embodiments the radio frequencies are 13.56 mHz for pairing with widely available readers, including smartphones. In the example below, the signal was large and the resistance of the poly(pyrrole) was lowered by 700,000% in response to physiological concentrations of glucose. Large changes (reductions) in resistance were used to create responses to pyruvate. The large resistivity changes observed can provide sensitivities that exceed conventional LFA technologies. In some embodiments, the analytes can be detected at concentrations lower than a nanogram/Liter. In some embodiments, the analytes can be detected at concentrations lower than 100 picograms/Liter. In some embodiments, the analytes can be detected at concentrations lower than 10 picograms/Liter.

The initial demonstration begins with the enzymes immobilized on the semiconducting polymer by action of an analyte, but the transduction scheme can be much broader. For example, the enzymes can be organized proximate to the semiconducting polymer and be effective as long as the action of the enzymes causes a change the resistivity of the semiconducting polymer. In some embodiments, a sample can be added to a reagent pad containing a receptor for the analyte bioconjugated to one of more enzymes, and other reagents that produce the oxidating of reducing products can be added or also present in the reagent pad. The reagent pad can be structured as to release these materials at the same time or in a staged fashion with one preceding the other in the flow along the assay. An assay can comprise two or more separate reagent pads, and in some embodiments one pad the sample containing an analyte is added and a second reagent pad a solution that delivers a second reagent for the assay. Additional reagent pads can deliver materials as needed. The time at which the different reagents are released can in some embodiments be controlled by the pathlength of the fluid flow, structuring of the reagent pad, or timing of the addition of fluids to the reagent pad. A multivalent analyte that is complementary to the receptor conjugated to the enzyme can become functionalized and then a second receptor attached to the semiconductive transducing material or the supporting structure can capture the analyte-enzyme complex upon flowing along the test strip. Enzyme substrates, including but not limited to substrates of oxidase enzymes including glucose or pyruvate, or reductase enzymes, can result in a local concentration of the oxidant or reductant that causes a resistance change in the control and test bands of the assay.

This scheme is general and can be applied for the detection of bacteria, proteins, viruses, nucleic acids, cells, reactive biomarkers, enzyme substrates, carbohydrates, a toxin, a heavy metal, an organic molecule, combinations thereof, or any analyte capable of binding to or reacting with an enzyme and any analyte capable of binding to two or more receptors at the same time. For example, a tetrameric C Reactive Protein (CRP) can be detected by using a test line with semiconductor functionalized with a CRP specific antibody and a reagent pad containing a glucose oxidase conjugated to a CRP specific antibody. Dimeric or monomeric proteins can be detected provided that two binding elements can interact with the protein. Multiple binding proteins can be used to attach to two separate epitopes of a protein analyte. In some embodiments, there can be advantages to using multiple binding proteins that recognize different epitopes. For example, if all of the target analytes are bound to one binding protein in the reagent pad, there can still be an open binding site (second epitope). Binding to this second epitope by a second binding protein can be used to localize an enzyme on or proximate to a semiconducting material.

By way of example, the semiconducting materials that can be used with this device include but are not limited to semiconducting polymers, conducting polymers, carbon nanotubes, graphene, carbon-nitride nanomaterials, metal oxides, MXenes, such as transition metal carbides or nitrides, or metal sulfides. In some embodiments, a first semiconductor can be doped which in turn can change the conductivity of a second semiconductor. In some embodiments, the second semiconductor can be silicon and in some embodiments the changes in the resistivity of the silicon can be used to determine the presence of an analyte. In other embodiments, the first semiconductor is not required to have high conductivity when doped. In some embodiments, the first semiconductor can be an isolated molecular form that is incapable of bulk semiconductive behavior.

In some circumstances, an LFA can make use of a change in resistivity in a semiconducting material for the quantitative detection of an analyte.

In other circumstances, an LFA can make use of a change in the resonance of a RFID circuit for the detection of an analyte.

In other circumstances, a method for deposition of semiconducting polymer films can involve the use of a colloidal dispersion, that provides a method for creating high quality conformal coatings. In some embodiments, the colloids can undergo reductive functionalization with a thiol or regent capable of doing a reaction equivalent to the thiol-Michael reaction, to create a film that is in a state optimal for oxidative doping. This coating and functionalization method can be inexpensive and is capable of being scaled for commercial production of LFAs as well as other coated materials.

In other circumstances, a method for depositing a semiconducting material can involve a solution that can be printed. In some embodiments, the printed material can be activated chemically or thermally after deposition to produce the transducing material. In other circumstances, the semiconducting material can be synthesized directly on the supporting material from molecular precursors. In some embodiments multiple materials can be deposited either together or adjacent to each other.

In other circumstances, a method of using a catalyst to facilitate the reaction of an oxidizing or reducing reagent with a semiconducting material can cause optimal conductivity changes. The redox catalyst can enhance or accelerate the change in the resistive state of the semiconductor with an analyte trigger.

In other circumstances, a quantitative LFA can be capable of being read wirelessly with a smartphone. In other circumstances, an inexpensive device capable of detecting a change in resistivity can read the LFA and transmit the information to a smartphone, local area network, cellular network, or computer.

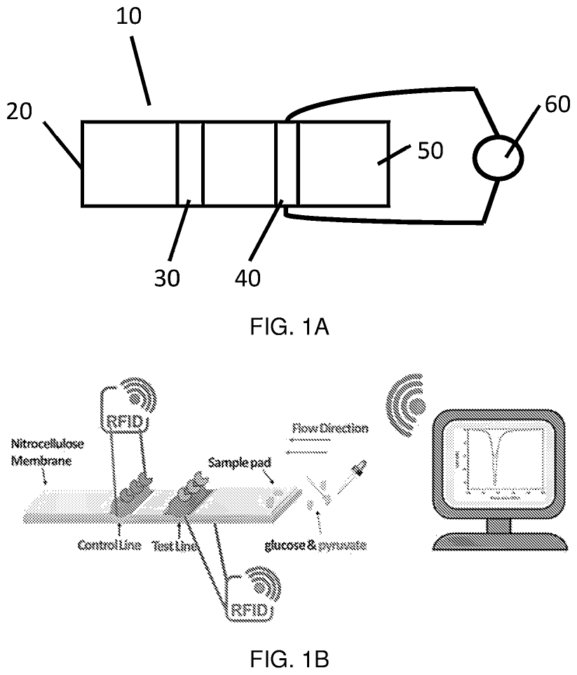

Referring to FIG. 1A, a device 10 can include a porous substrate 20. The porous substrate can be capable of transporting sample through capillary action. The porous substrate 20 is arranged to receive a fluid sample, for example, at sampling region 50. A fluid sample can be delivered to sampling region 50 as a drop or as a flow of fluid. The sampling region can contain reagents and recognition moiety necessary for the assay. A control region 30 can be present on the porous substrate 20. The control region can provide a visual or electronic signal confirming presence of the fluid sample on the device. A sensing region 40 can be present on the porous substrate 20 and can be adjacent to control region 30. The sensing region 40 can include a semiconducting material that has an electronic property that changes in response to the analyte. The electronic property can be monitored by a circuit 60. The electronic property in the control region 30 can also be measured by an equivalent circuit 60 (not shown). The control 3, and sensing 40 regions need not be in the order shown and in some embodiments the control region can be before the sensing region. The device is also not limited to one sensing region and a plurality of sensing regions can be used to create an LFA capable of detecting multiple analytes from a single sample. Here again multiple circuits 60 can be connected to each sensing region. The sensing regions can be wired separately of in parallel depending on the intent of the assay. The electronic property can be resistivity of the sensing region.

The fluid sample can have a volume of 5 nL, 10 nL, 50 nL, 100 nL, 500 nL, 1 microL, 5 microL, 10 microL, 20 microL, 50 microL, 100 microL, 500 microL, or 1 mL. The sample can be in a liquid, for example, water, a water solution containing molecules and/or ions, or an organic solvent such as an alcohol, an ether, or an ester, or combinations thereof.

The semiconducting material can include a semiconducting polymer film on the surface of the porous substrate. The semiconducting polymer film can include a polypyrrole, a polythiophene, a polyacetylene, a polyphenylene, a polyphenylene vinylene, or another semiconducting polymer. In certain embodiments, the semiconducting polymer film initially can be in a low conductive state. In certain embodiments, the semiconducting polymer film is initially in a high conductive state.

The porous substrate can be a fibrous structure, a foam structure, a matted structure, or a non-matted structure. The porous substrate permits a fluid sample to flow toward the sensing region. In other embodiments, the porous substrate is a filter that a fluid can pass through. The porous substrate can include paper, cotton, polyester, glass, nylon, mixed cellulose ester, spun polyethylene, polysulfone, nitrocellulose, nylon, a poly(arylene ether), or mixed cellulose ester. The porous substrate can have an average pore diameter of 50 microns, 60 microns, 70 microns, 80 microns, 90 microns, 100 microns, 110 microns, 120 microns, 130 microns, 140 microns, 150 microns, 160 microns, 170 microns, 180 microns, 190 microns, 200 microns, 210 microns, 220 microns, 230 microns, 240 microns, or 250 microns. The porous structure can have a thickness of 0.01 mm, 0.1 mm, 0.2 mm, 0.4 mm, 0.5 mm, 0.6 mm, 0.7 mm, 0.8 mm, 0.9 mm, 1.0 mm, 1.1 mm, 1.2 mm, 1.3 mm, 1.4 mm, 1.5 mm, 1.6 mm, 1.7 mm, 1.8 mm, 1.9 mm, 2.0 mm, 2.1 mm, 2.2 mm, 2.3 mm, 2.4 mm, or 2.5 mm. When coated, the average pore diameter can be reduced by 20%, 30%, 40%, 50%, 60%, 70%, or 80%. The porous substrate can be designed to transport the analyte and key reagents in the LFA and that other structured materials based on polymers, biomaterials, or inorganic materials could have equivalent performance and be used in these devices.

In some embodiments, the porous material can be designed to reduce the speed that the solution flows by the semiconducting material to allow for greater time for redox reactions. Extra time can result in higher sensitivity. This feature can be a direct effect of the change in the character of the porous material as a result of deposition of the semiconducting materials. For example, if the semiconducting material reduces the interaction with the water or reduces the pore size it can slow the passage of water. Alternatively, the porous substrate can be deposited proximate to the semiconducting material to slow fluid flow passing the semiconducting material.

The semiconducting material, or a material proximate to the semiconducting material, can include a recognition moiety configured to respond to the analyte. The recognition moiety can selectively bond with or otherwise identify the analyte. For example, a probe can bind to the recognition moiety that oxidizes or reduces the analyte. In other examples, the analyte can itself be a catalyst for oxidization of reduction processes. In certain circumstances, a catalyst can be present to oxidize or reduce the analyte. The catalyst can, in some embodiments, facilitate the reaction of an oxidizing reagent with a semiconducting material to cause optimal conductivity changes. Other specific interactions can trigger conductivity changes, including metal binding, protonation, or other reactions. The catalyst can be a metal catalyst, for example a metal oxide such as a molybdate. Alternatively, the catalyst can include an enzyme for which the analyte is a substrate. In certain circumstances, a catalyst and an enzyme can both be present in the sensing region. For example, the analyte can be capable of binding to or reacting with an enzyme or capable of binding to two or more receptors at the same time. In some embodiments the analyte binds to one recognition moiety in the reagent pad and a second recognition moiety in the sensing region. In some embodiments the first recognition moiety contains an enzyme. The recognition moieties can be antibody-enzyme conjugates, protein-enzyme conjugates, conducting polymer-protein conjugates, conducting polymer antibody conjugates. These are non-limiting examples intended to illustrate how an analyte that binds to 2 or more recognition moieties can be used to colocalize materials at the sensing region. In come embodiments one or two more reagents added that do not interact with the analyte directly, but react with the analyte assembled complex localized at the sensing region to produce a change in the electronic property of the semiconducting material. The recognition moiety can include an enzyme, protein, synthetic receptor, antibody, nano-body, nucleic acid, molecular catalyst, metal binding site, or combinations thereof.

A method of manufacturing a device described herein can include depositing a colloidal dispersion on a surface of a substrate to form a semiconducting material. The colloidal dispersion can include particles capable having fluidic properties that a subsequently ruptured on the surface of the substrate to form a substantially uniform film. The colloidal dispersion can be functionalized such that the resulting film has chemical or biological function. For example, the colloids can be connected to recognition moieties prior to forming a film and the film can present these moieties in a way that can recognize analyte. Alternatively, in other embodiments the colloidal dispersion will be functionalized such that the film formed has surface functionality that can be used for subsequent functionalization. The semiconducting material can be deposited from solution to create structures on a substrate by spray coating, silk screen printing, ink jet printing, blade coating, or other physical deposition method, or combinations thereof. In some embodiments, the semiconducting materials can be soluble and deposited from solution. In some embodiments, the deposited material is a precursor to a semiconducting material that is then activated chemically or photochemically. In other embodiments, a semiconducting material is assembled or synthesized directly on the substrate. For example, a polymer can be produced on the substrate by polymerization of a monomer.

In certain embodiments, the surface can be a rough surface.

In certain embodiments, the surface can be porous.

In certain embodiments, the surface can be fibrous.

In certain embodiments, the surface can be cloth.

In certain embodiments, the surface can be a metal.

In certain embodiments, the surface can be a metal oxide.

In certain embodiments, the surface can be a filter.

In certain embodiments, the substrate can include a collection of particles.

In certain embodiment, the substrate can be composite. For example, the substrate can be a composite of one or more of the materials mentioned herein.

Analyte detection refers to the ability to confirm the presence of an analyte at a concentration of interest for a given application. In some embodiments, the concentration can be millimolar. In some embodiments, the concentration can be micromolar. In some embodiments, the concentration can be nanomolar. In some embodiments, the concentration can be picomolar. In some embodiments, the concentration can be femtomolar. The detection signal can be larger than the background noise. The higher the signal relative to noise, the greater accuracy possible to determine the concentration of the analyte in the analyte detection process. As a result, larger resistance changes in the analyte detection process can, in some embodiments, provide for higher accuracy in determining the concentration of an analyte.

Each of the substrate and the semiconducting material, independently, can be modified by surface chemistry reactions applied at different stages. For example, reductive functionalization of the semiconducting material assembled in a colloidal dispersion with a thiol or regent capable of doing a reaction equivalent to the thiol-Michael reaction, can create a material capable of forming film that is in a state optimal for analyte detection by for oxidative doping. This method can be inexpensive and capable of being scaled for commercial production of LFAs. In other embodiments the thiol-Michael reaction can also be formed on the material after forming the film. The thiol-Michael reaction can be used to directly connect a recognition moiety or to provide other functional groups that can be used to connect the recognition moiety in a later step.

In other circumstances, the substrate upon which the semiconducting material is located can be functionalized. This functionalization can be done before or after deposition of the semiconducting material. The functionalization of the substrate can be performed proximate to the semiconducting material. In some embodiments, both the substrate and the semiconducting materials can be reacted to create include the same or different functional groups. The functionalization chemistry can be selected as is appropriate for the functionality and substrate reactivity. In some embodiments, the multiple functionalization steps can be used. For example, in one step, a functional group may be added that allows for facile attachment of another group. In some embodiments, a first functionalization can attach a reactive ester that can then be reacted with a biomolecule, which in some cases could be reacted with yet another biological molecule to produce a final structure.

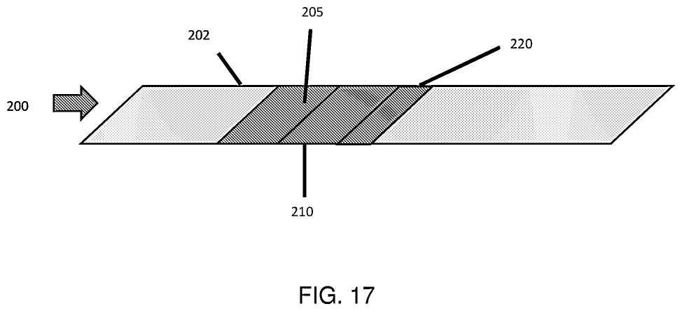

The sensing region can have multiple sub-regions including regions that can have recognition moieties or recognition elements, regions that can include semiconducting materials, and, optionally or in combination, regions that can have functionality that is designed to control the rate of fluid flow. The sensing region components can be optionally co-localized or adjacent to each other. The ordering of the regions can reflect the direction of fluid flow. For example, an adjacent group that slows fluid flow may be put down-stream of the semiconducting material, but a recognition moiety that binds to analyte and causes a change in the semiconducting material can be in the same location as the semiconducting material or up-stream. Referring to FIG. 17, a device 202 having a direction of fluid flow 200 can include a recognition zone 205, a semiconductor material zone 210 and a flow modifier zone 220. The recognition zone 205 can bind an analyte and catalysis can create a reactant that changes conductivity. The semiconductor material zone 210 can include a feature that may bind an analyte. The flow modifier zone 220 can slow the flow of liquid media past the semiconductor material zone.

In some embodiments, the sensing region can be relatively narrow relative to the flow of the solution in the lateral flow assay. The sensing region can have a width of 0.01 mm, 0.1 mm, 0.2 mm, 0.4 mm, 0.5 mm, 0.6 mm, 0.7 mm, 0.8 mm, 0.9 mm, 1.0 mm, 1.1 mm, 1.2 mm, 1.3 mm, 1.4 mm, 1.5 mm, 1.6 mm, 1.7 mm, 1.8 mm, 1.9 mm, 2.0 mm, 2.1 mm, 2.2 mm, 2.3 mm, 2.4 mm, or 2.5 mm. In some embodiments, the sensing region can be wider that 2.5 mm. The desired specific width of the sensing region can be selected based on one or more of the following features: the manufacturing method; the desired base resistivity of the sensing material before and after a sensing event; the need to make electrical contacts; the size of the support; the nature of the reactions that cause changes in the semiconducting material; and the ease with which an electrical measurement can be made.

The circuit 60 can be a resistivity sensor. In other embodiments, circuit 60 can be a radio frequency identification tag electrically connected to the sensing region 40. For example, the radio frequency identification (RFID) tag can include an integrated circuit in parallel or in series with the sensing region. The electronic property of the sensing region can be measured, for example the frequency of the RFID tag, or monitored by a reader, such as a wireless reader. The wireless reader can be, for example, a loop antenna or a smartphone or any circuit capable of monitoring a property of the sensing region, which can be calibrated to determine the concentration of the analyte in the sample.

The analyte can include a bacterium, a protein, a virus, a nucleic acid, a cell, a reactive biomarker, an enzyme substrate, a carbohydrate, a toxin, a heavy metal, an organic molecule, or combinations thereof.

An example of the system including the device and a detector is shown in FIG. 1B. The system can carry out a method of detecting an analyte that can include exposing the device to a sample, and detecting the analyte in the sample. The step of detecting the analyte in the sample can be quantitative. For example, the reader can be calibrated to provide a quantitative measure of the analyte. A quantitative assay can be used to determine biomarkers that are always present, but when their concentration is out of a normal range, they can indicate a health issue.

Herein, the design of a wireless lateral flow device and demonstrate the conversion of oxidase reactions to changes in the resonance of radiofrequency identification (RFID) circuits is reported. The detection is triggered by large conductivity changes induced by a polyoxometalate-catalyzed oxidative doping of polypyrrole (pPy) when exposed to oxidase-generated H₂O₂. This transduction and RFID capability has been integrated into a lateral flow device to create a low-cost, rapid, and portable method for quantitative biological signal detection. In addition, a new method for creating functional coatings from pPy core-shell colloidal particles bioconjugated using streptavidin-biotin recognition with glucose oxidase or pyruvate oxidase is reported. The bio-functionalized pPy particles coalesce on the nitrocellulose membrane to produce a chemiresistive band. Addition of glucose or pyruvate solutions results in formation of H₂O₂ at the pPy bands, functionalized with the respective oxidase, to produce conductivity enhancements exceeding 700,000%. Placing the pPy band within the RFID circuit converts the changes in resistivity to the changes of resonance. The detection of enzymatic response of glucose oxidase within 30 mins with as low as 0.01 wt % of glucose using this lateral flow device was achieved. Pyruvate was also shown to produce remarkable responses. The ability to use oxidase enzymes as transduction elements establishes this method as a platform for the construction of a new family of lateral flow devices capable of detecting and quantifying biological targets. The oxidase enzyme transduction is one non-limiting example of the method and sensors disclosed herein.

The production and detection of biochemical signals is integral to all biological systems (Refs. 1-3), and often involves enzyme-catalyzed reactions (Refs. 4-8). The present understanding of biochemistry has revealed numerous coupled systems (Refs. 7, 9-11). Glucose oxidase is a very robust enzyme that is widely distributed in living organisms and with oxidation of glucose produces hydrogen peroxide (H₂O₂) (Refs. 12-14). In addition to its utility in glucose detection, this enzyme also finds applications in wound healing, food preservation, and pharmaceutical industry (Refs. 15-17). Glucose oxidase can be used in colorimetric glucose assays by converting the production of H₂O₂ to the absorbance (color) changes (Refs. 18-19). The generated H₂O₂ can also be detected electrochemically and this method is the basis of conventional glucose monitoring devices (Refs. 20-22). The robust and versatile nature of glucose oxidase qualifies it as an attractive platform for the creation of additional biosensor platforms. In some embodiments, the analyte can be itself a catalyst of a reagent for a transduction event that provides a sensing response. In other embodiments, a reductase enzyme can be used to create a transduction event.

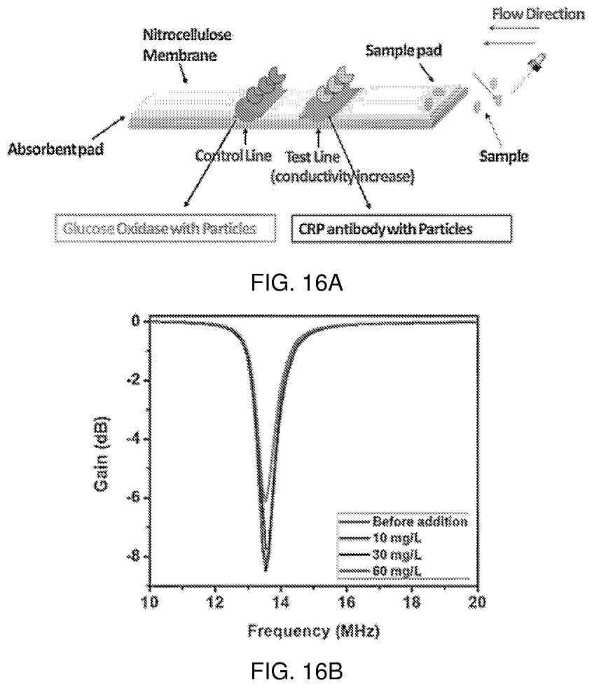

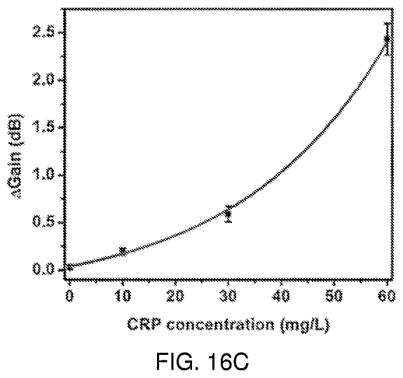

Lateral flow assays (LFAs) have emerged as an economical, fast, and easy to use platform for laboratory medical diagnostics and home testing (Refs. 23-26). In an LFA, liquid flow driven by capillary force moves samples and reagents along a nitrocellulose test strip (Refs. 27-28). The sample pad is loaded with capture reagents that are designed to produce signals at test and control lines and have immobilized specific biological capture agents. A limitation of conventional colorimetric LFAs is that most provide a binary (yes/no) readout rather than quantitative data (Refs. 26, 29). As a result, a sensitive LFA platform has been created that can be readily used to create quantitative data. As an example, C-reactive protein (CRP) is an inflammation biomarker tetrameric protein that has normal ranges in humans, and will increase in response will be produced in response to an immune response. FIGS. 16A-16C show that this biomarker can be quantatitively detected when the test line has an organic semiconductor polymer (polypyrrole) functionalized with antibodies that specifically bind to CRP, the reagent pad has a CRP specific antibody conjugated to a glucose oxidase, and the solution contains glucose. Such a device can be used to monitor, for example, the health of an individual undergoing immunotherapy for cancer treatment.

As described herein, carboxylate-functionalized pPy core-shell particles are used to create oxidase conjugated films for signal transduction. The functionalization of the pPy creates a reduced low conductivity state that can be coated to give a resistive band on the LFA test strip. The pPy bands can be oxidatively doped by H₂O₂ to give more than a 700,000% increase in conductivity. These large changes in resistance can be determined by integration into a resonant radiofrequency identification (RFID) circuit, or can be measured directly by four-point probe or two-point probe methods, and thereby determine glucose or pyruvate concentrations (Refs. 30-35). The RFID method of detection using passive near-field communication (NFC) tags operating at 13.56 MHz has been demonstrated herein. These devices can be powered and read by conventional smartphones for convenient home testing (Refs. 36-39).

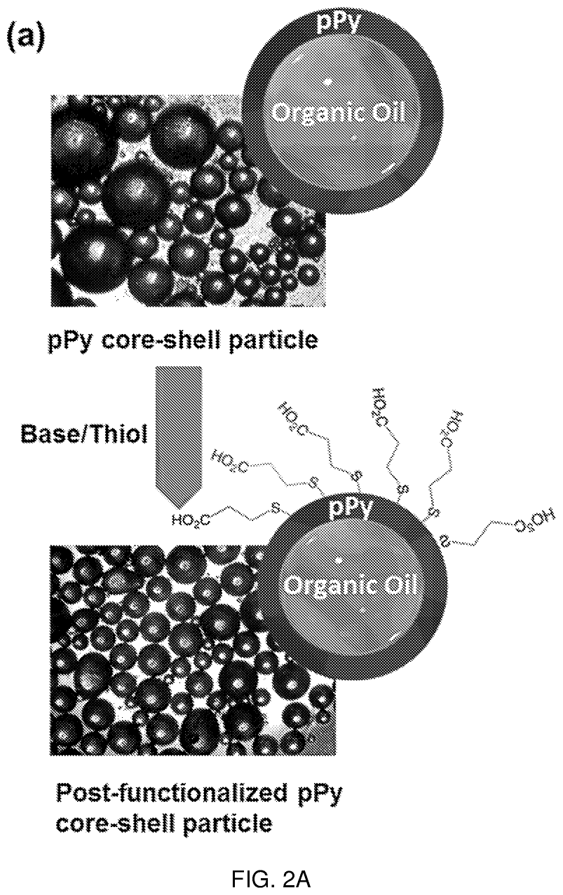

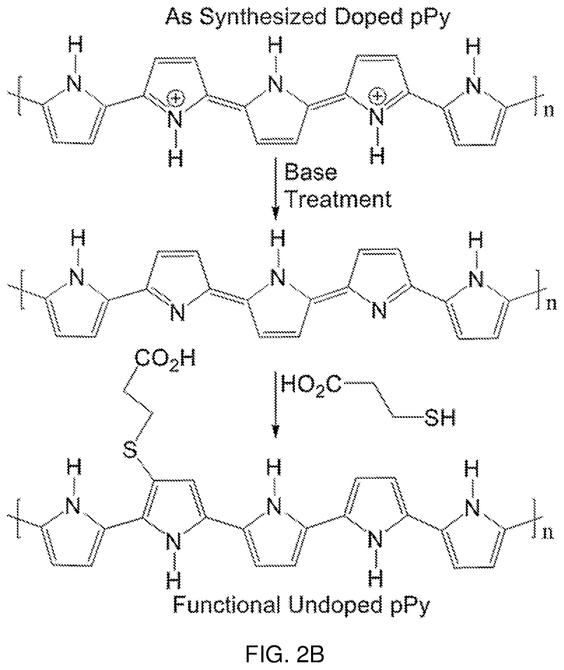

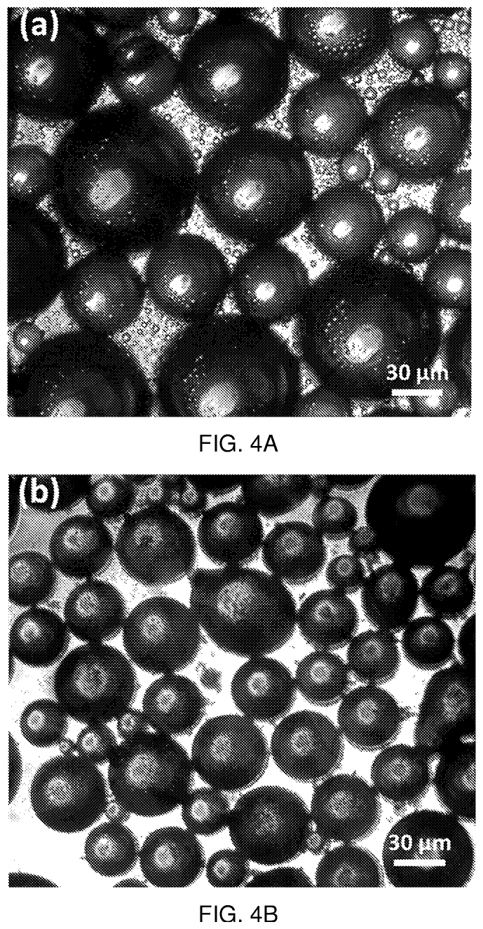

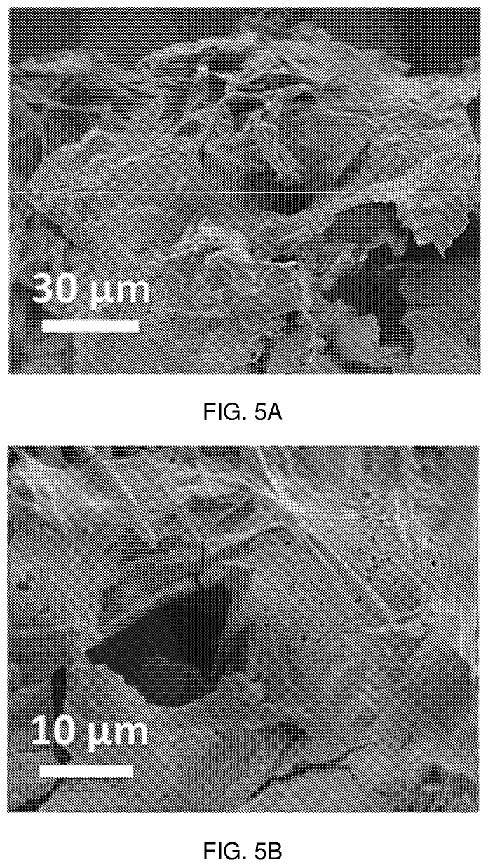

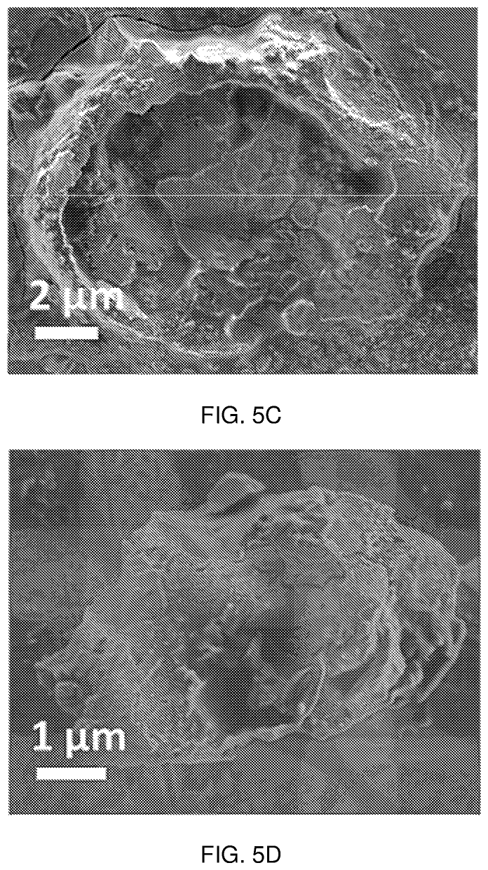

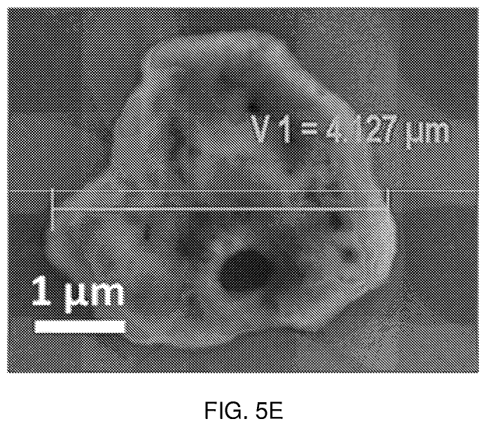

Efficient fabrication methods compatible with large-scale production of the LFA test strips are necessary for real-world applications. To this end, a new method has been developed that makes use of colloid dispersions that contain insoluble conducting polymers that are produced at a liquid-liquid interface. These materials have fluid-like properties and can be used to produce high quality conformal coatings on a variety of substrates, including porous materials The method also is ideally suited for facile functionalization and bioconjugated pPy or other semiconductor particles can be produced as activated colloids to conformally coat nitrocellulose membranes. As detailed in FIGS. 2A-2B, FIGS. 3A-3B and FIGS. 4A-4B, this method makes use of core-shell particles produced by interfacial emulsion polymerization of organic-pyrrole droplets in water. The initial core shell particles contain oxidized (doped) pPy and if deposited directly on surfaces produce create conductive coatings. The thin pPy shells and the organic core solvent produce reproducible method for depositing a uniform coating. However, in our present application to create the necessary transduction lines for our LFA devices, the pPy needs to be in a highly resistive (undoped) state that can optimally give the largest conductance response to an oxidation (doping) event. To meet this design requirement, a method to accomplish both conjugation and reduction in one procedure (FIGS. 2A-2B) has been developed. Specifically, the free-base form of the doped pPy is generated in situ and readily functionalized by a thiol-Michael reaction with a functional-thiol. After tautomerization, the resultant state shown in FIG. 2B is an undoped polymer. 3-mercaptopropionic acid has been used as the functional-thiol for addition and the carboxylate group can be used for bioconjugation (FIGS. 5A-5E). These materials can be coated on glass as well as nitrocellulose and with higher concentrations. This and similar methods can be used to efficiently synthesize, functionalize, and coat a variety of semiconductive materials including polythiophenes and polyanilines. Other manufacturing methods can be used that employ soluble semiconducting materials or precursors.

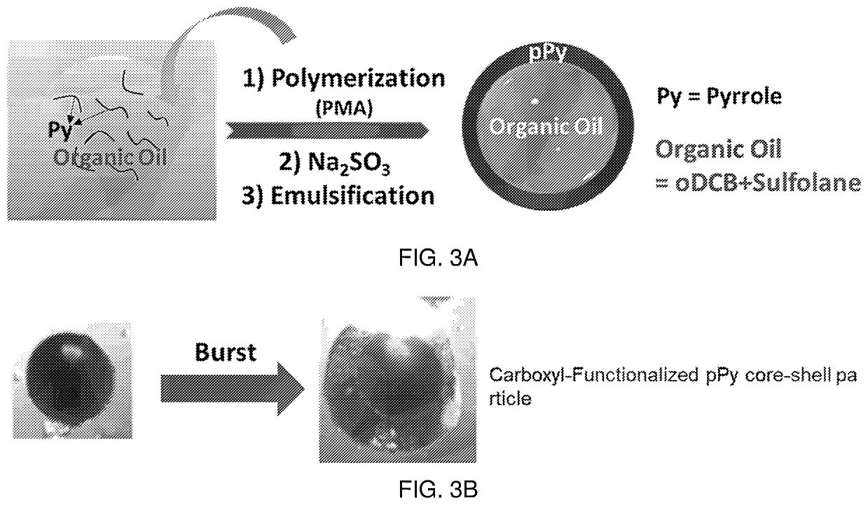

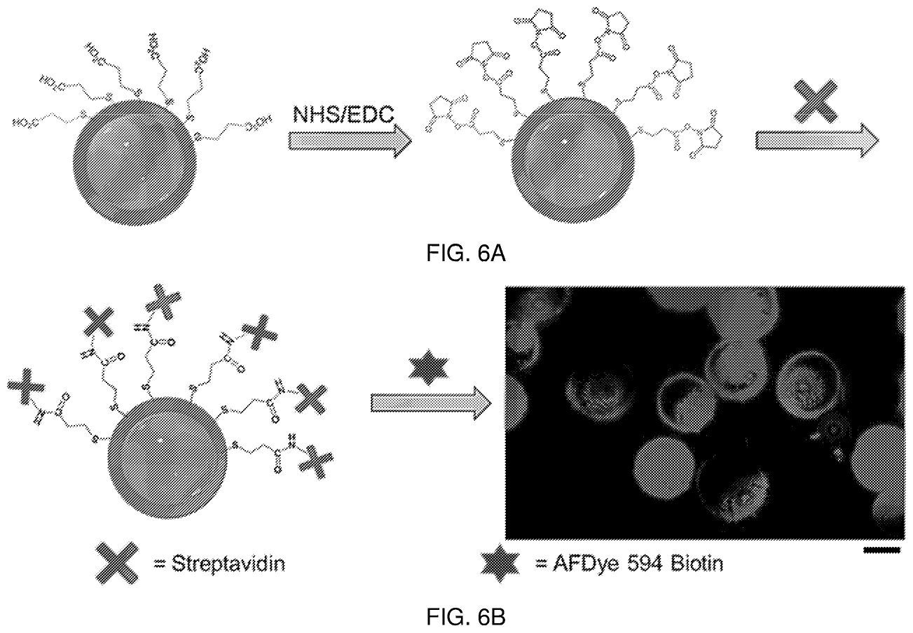

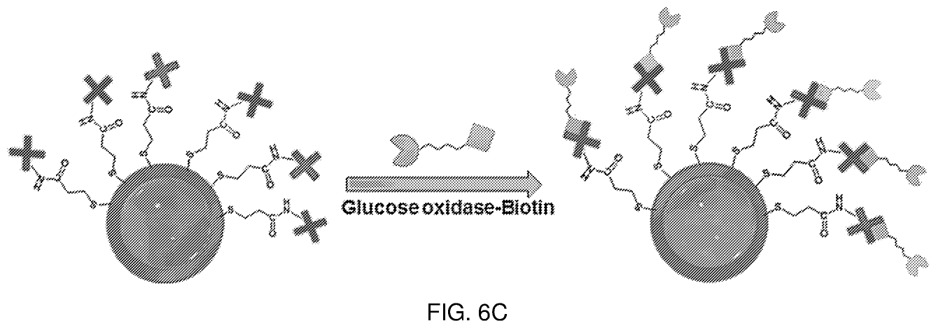

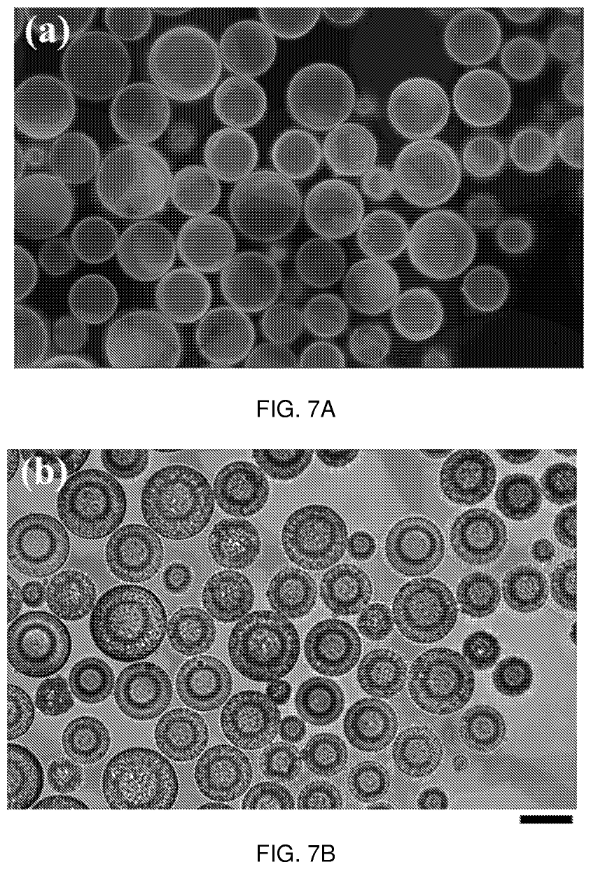

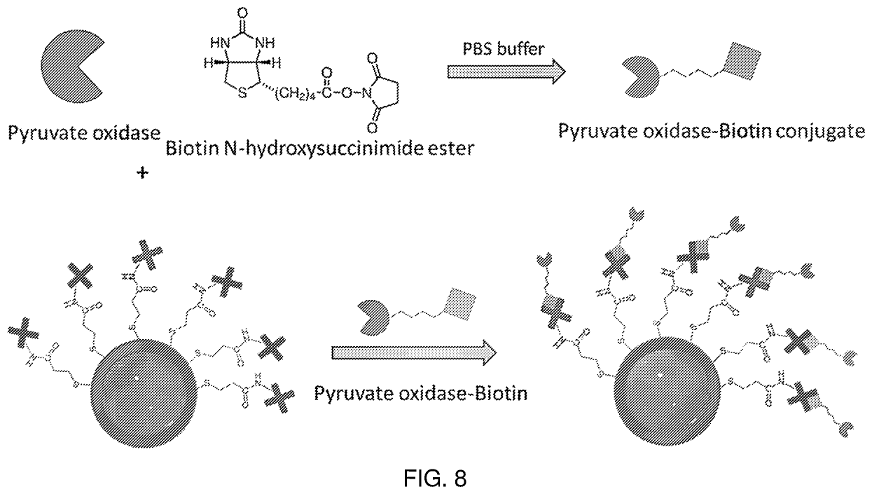

The carboxylate-functionalized pPy core-shell particles were further bio-conjugated via an NHS ester activated condensation reaction (FIGS. 6A-3C and FIGS. 7A-7B, and 8). In this process, carboxyl groups were treated with N-hydroxysuccinimide and a carbodiimide to create NHS esters that reacted with streptavidin's amines. The streptavidin-pPy core-shell particles were then bound to biotinylated glucose oxidase or pyruvate oxidase. An advantage of the streptavidin/biotin conjugation scheme is that streptavidin's tetravalency can in principle create a higher density of enzymes at the surface of the pPy core-shell particles. Fluorescent microscopy (FIG. 6C) confirms the streptavidin conjugation by staining with a red fluorescent biotin-dye conjugate.

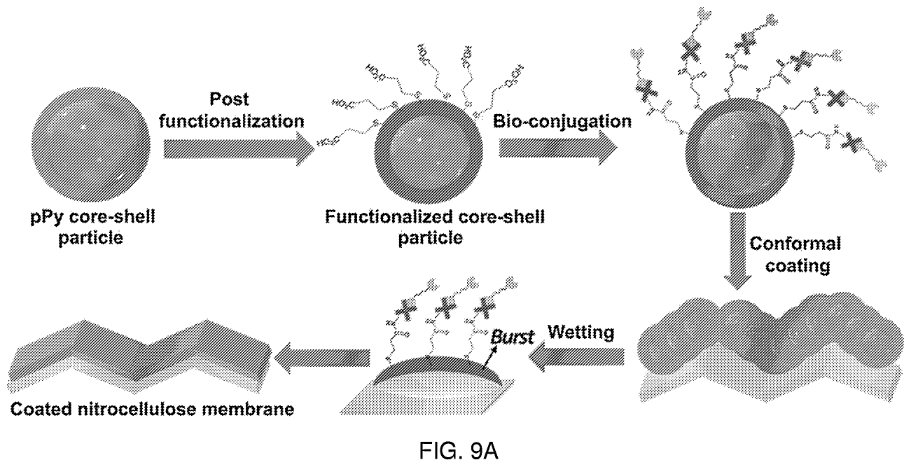

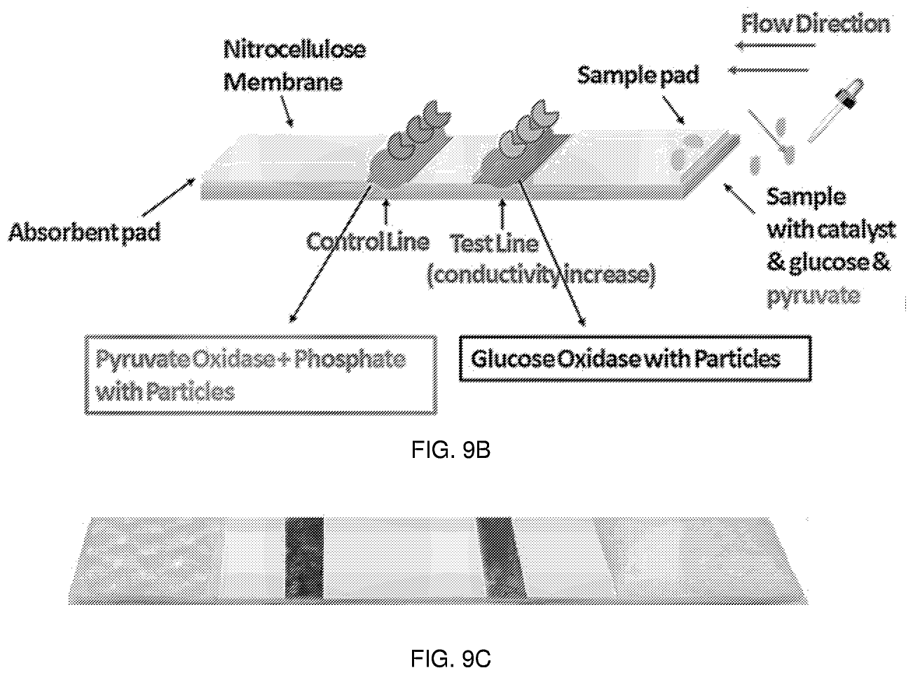

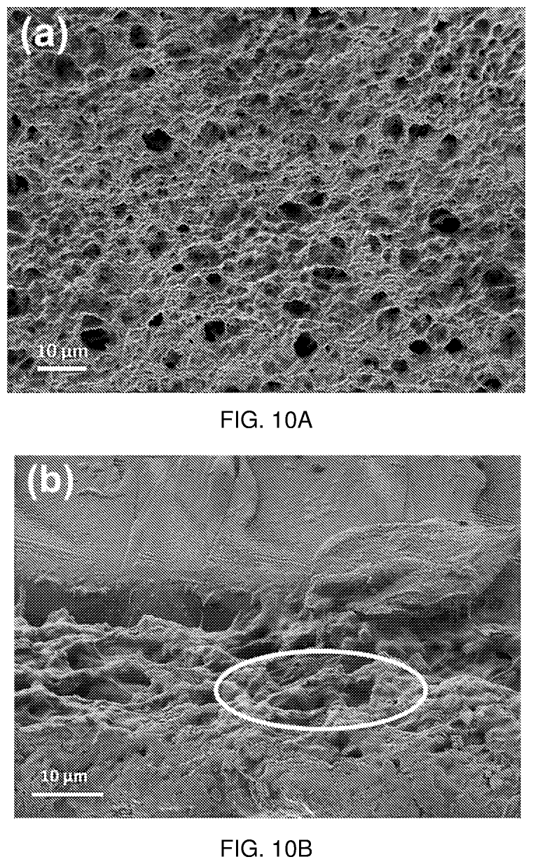

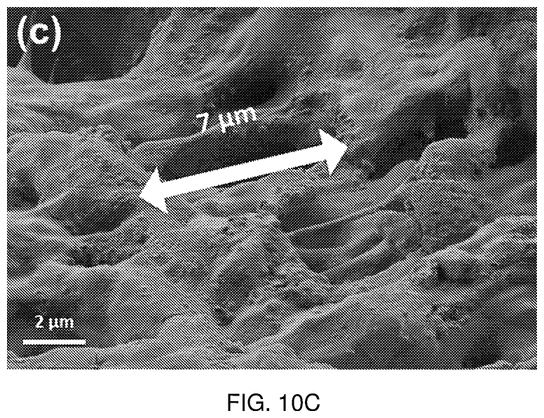

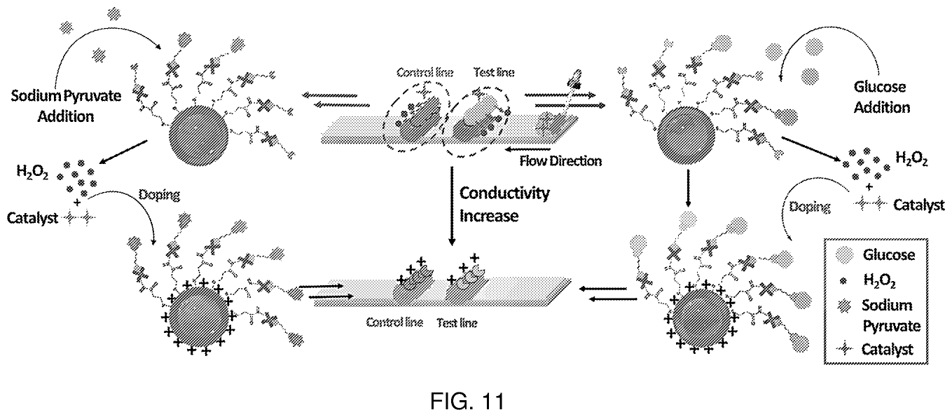

The bio-conjugated pPy core-shell particles were directly coated on nitrocellulose test strips for use in an LFA (FIGS. 9A-9C). Scanning electron microscope images shown in FIGS. 10A-10C confirm that the pPy coated nitrocellulose test strips retain their porosity with 7 μm pores. For a LFA to be effective, fluid must flow over both test and control lines, which are respectively functionalized by pPy films with surface glucose and pyruvate oxidase enzymes. The nitrocellulose is generally functionalized with surfactants to enable a hydrophilic surface to promote lateral capillary flow of aqueous sample solutions (Ref. 40). The hydrophilic nature of the bioconjugated pPy films deposited appear to function similarly to the surfactants and do not impede this necessary fluid flow. The device has been configured with a glucose-oxidase-pPy test line and a pyruvate-oxidase-pPy control line. These enzymes generate H₂O₂ that can be used to oxidatively dope the pPy and create a conductivity change that is determined by the amount of glucose and/or pyruvate present (FIG. 11). In such a device, a known amount of pyruvate added as a control to ensure the reliability of the LFA.

TABLE 1
Summary of conductivity before and after the application of glucose solutions to the
lateral flow device. The redox catalyst is phosphovanadomolybdate (H₅[PV₂Mo₁₀O₄₀]).

| | Glu/Cat. Conc. | | | | | | |
|---|---|---|---|---|---|---|---|
| | 0.2 wt % | | 0.05 wt % | | 0.01 wt % | | Glucose-Free |
| | Glucose | | Glucose | | Glucose | | Water |
| | 0.16 wt % Cat. | | 0.16 wt % Cat. | | 0.16 wt % Cat. | | 0.16 wt % Cat. |
| Before | 2.969*10⁻⁴ | S/cm | 5.485*10⁻³ | S/cm | 4.140 *10⁻⁴ | S/cm | 8.858*10⁻³ S/cm |
| Addition | | | | | | | |
| After | 2.195 | S/cm | 0.122 | S/cm | 3.620*10⁻³ | S/cm | 4.954*10⁻³ S/cm |
| Addition | | | | | | | |
| Conductivity | 7394 | times | 204 | times | 8 | times | No Increase |
| Enhancement | | | | | | | |

Using the nitrocellulose strips containing bio-conjugated pPy test and control lines, the lateral flow device has been assembled as shown in FIGS. 9B and 9C. Application of liquid samples containing glucose, a redox catalyst, and sodium pyruvate resulted in transport of solutions across the test and control lines. Glucose and pyruvate oxidase catalyzed oxidation resulted in the formation of H₂O₂ to dope the pPy film and cause a conductivity increase. The changes of conductivity on the pPy film coated on the nitrocellulose membrane were first quantified by four-point probe method. It was determined that the intrinsic reactivity of H₂O₂ with pPy was too slow and hence a phosphovanadomolybdate (H₅[PV₂Mo₁₀O₄₀]) was used as a water-soluble oxidation (redox) catalyst that has been shown to be compatible with oxidases (Refs. 41-42). Table 1 and Table 3 detail the conductivity changes observed with different concentrations of glucose or sodium pyruvate. Optimized experimental conditions revealed that 0.16 wt % of catalyst provides the best conductivity enhancement (Table 2). LFA analysis of solutions containing 0.2 wt % of glucose and 0.16 wt % of catalyst produced a 7000-fold increase in conductivity. Using the optimized catalyst, solutions were also tested containing 0.05 wt %, 0.01 wt % of glucose and conductivity enhancements of 204- and 8-fold were observed, respectively. These results confirm that with a glucose trigger, dramatic increases in conductivity of the pPy film could be observed. The studies here were conducted with glucose concentrations at physiological levels or below, and with higher concentrations of glucose even larger enhancements of conductivity can be expected. It is noteworthy that heavily oxidized pPy often displays conductivity in excess of 200 S/cm and the highest value in Table 1 is about 100 times lower than this limit. Control tests wherein a solution is applied without the addition of glucose in the system resulted in no increase in conductivity (Table 4) indicating that the conductivity of pPy film is stable to background conditions. A two-point probe method (Table 5) was utilized, which is a more convenient method for measuring the conductivity changes for the lateral flow device.

TABLE 2
Summary of conductivity enhancements before and after
the application of sample solutions with different concentrations
of catalyst to the lateral flow device.

| | Glu/Cat. Conc. | | | | | |
|---|---|---|---|---|---|---|
| | 2 wt % | | 2 wt % | | 2 wt % | |
| | Glucose | | Glucose | | Glucose | |
| | No Cat. | | 0.08 wt % Cat. | | 0.16 wt % Cat. | |
| Before | 2.06*10⁻⁴ | S/cm | 2.49*10⁻⁴ | S/cm | 4.19*10⁻⁴ | S/cm |
| Addition | | | | | | |
| After | 0.018 | S/cm | 0.0628 | S/cm | 4.349 | S/cm |
| Addition | | | | | | |
| Conductivity | 88 | times | 252 | times | 10376 | times |
| Enhancement | | | | | | |

TABLE 3
Summary of conductivity enhancements before and after the application
of sample solutions to the lateral flow device film coated by
pPy particles bioconjugated with pyruvate oxidase.

| | |
|---|---|
| 0.2 wt % Glucose | No Glucose |
| 1 wt % pyruvate | 1 wt % pyruvate |
| 0.16 wt % Cat. | 0.16 wt % Cat. |

| | | | | |
|---|---|---|---|---|
| Before Addition | 7.220*10⁻⁴ | S/cm | 2.910*10⁻⁴ | S/cm |
| After Addition | 0.569 | S/cm | 0.289 | S/cm |
| Conductivity | 788 | times | 933 | times |
| Enhancement | | | | |

TABLE 4
Summary of the Conductivity enhancements measured by four-point
probe after being treated by different concentrations of glucose
or sodium pyruvate. All contain 0.16 wt % of catalyst, n = 3.

| Glu/Cat. Conc. | Conductivity Enhancement |
|---|---|
| 0.2 wt % Glucose | 7656 ± 2000 |
| Glucose Oxidase | |
| 0.05 wt % Glucose | 458 ± 200 |
| Glucose Oxidase | |
| 0.01 wt % Glucose | 8 ± 3 |
| Glucose Oxidase | |
| No Glucose | 1.6 ± 1.3 |
| Glucose Oxidase | |
| 1 wt % Sodium Pyruvate/0.2 wt % | 564 ± 150 |
| Glucose Pyruvate Oxidase | |
| 1 wt % Sodium Pyruvate/No | 672 ± 230 |
| Glucose Pyruvate Oxidase | |

TABLE 5
Summary of the Conductivity enhancements measured by two-point
probe after being treated by different concentrations of glucose
or sodium pyruvate. All contain 0.16 wt % of catalyst, n = 3.

| Glu/Cat. Conc. | Conductivity Enhancement |
|---|---|
| 0.2 wt % Glucose | 245 ± 100 |
| Glucose Oxidase | |
| 0.05 wt % Glucose | 18 ± 4 |
| Glucose Oxidase | |
| 0.01 wt % Glucose | 2.6 ± 0.4 |
| Glucose Oxidase | |
| No Glucose | 0.6 ± 1.4 |
| Glucose Oxidase | |
| 1 wt % Sodium Pyruvate/0.2 wt % | 549 ± 100 |
| Glucose Pyruvate Oxidase | |
| 1 wt % Sodium Pyruvate/No | 207 ± 50 |
| Glucose Pyruvate Oxidase | |

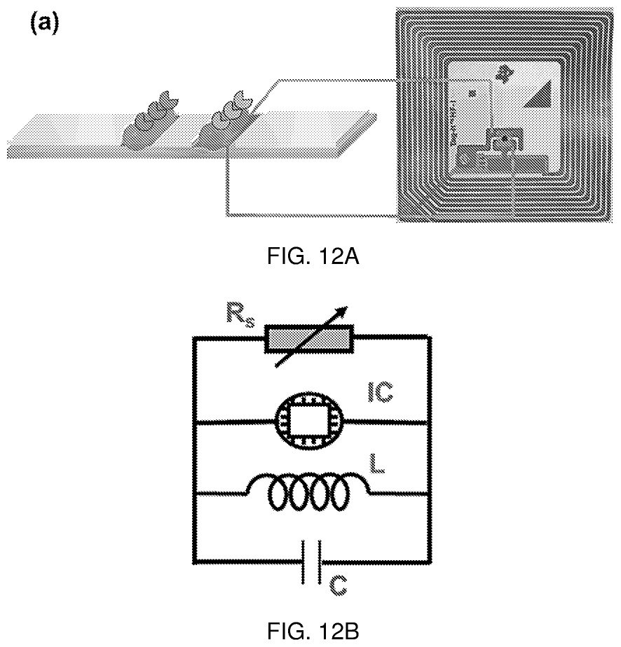

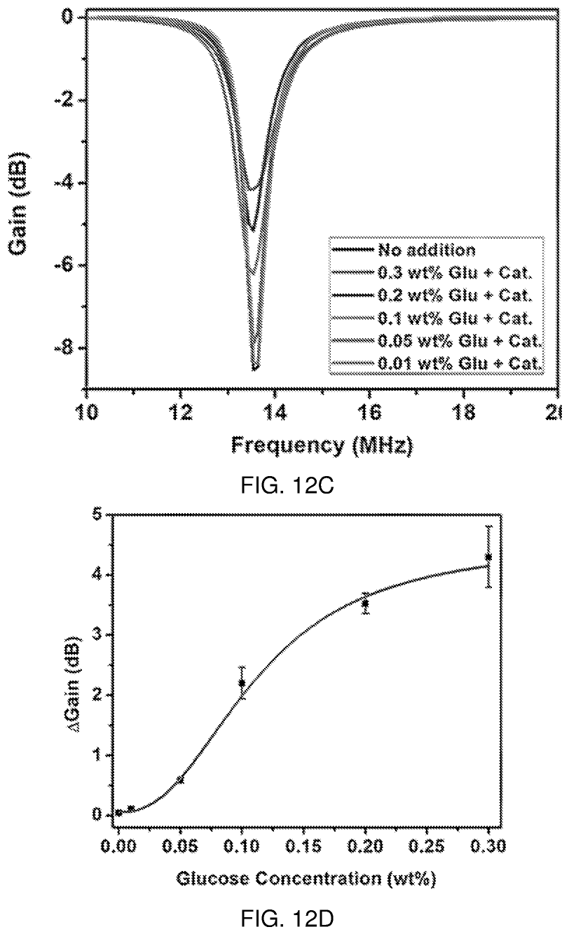

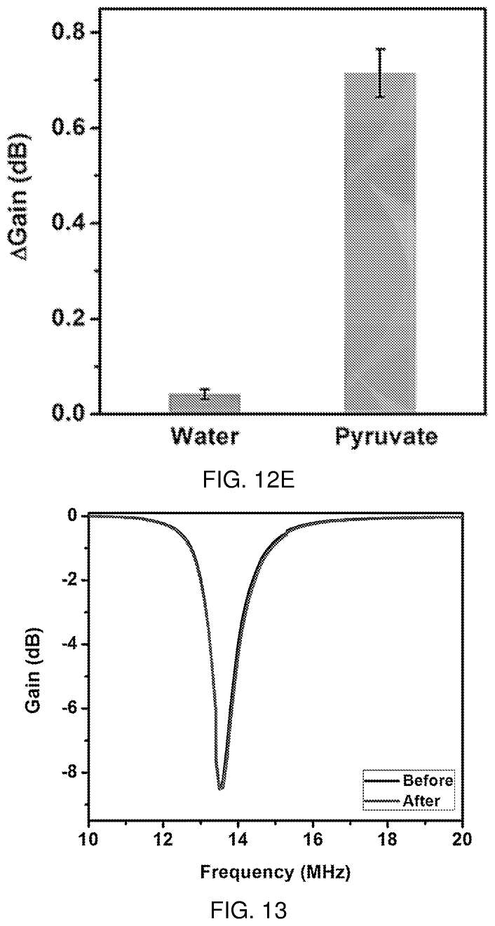

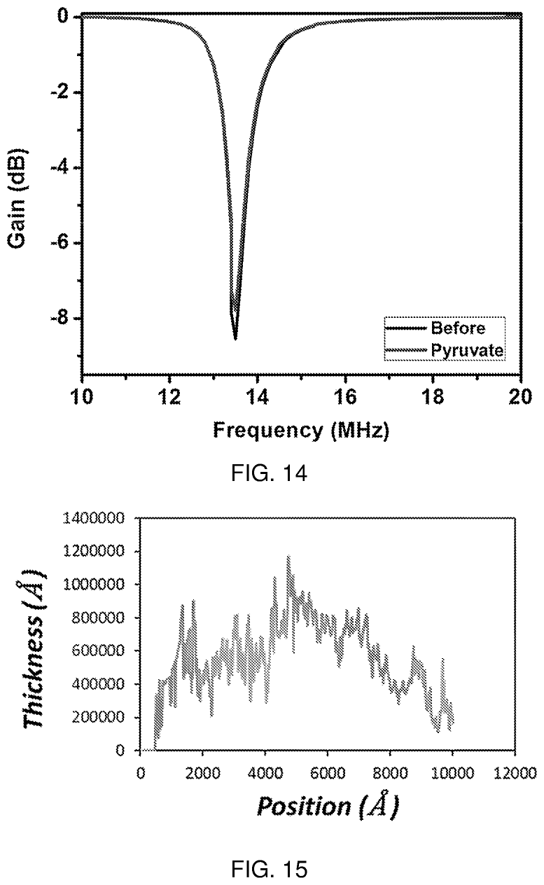

RFID devices have been widely used to track and identify the commercial goods using wireless communication. A single step conversion of near-field RFID tags into chemically actuated resonant devices (CARDs) and when the chemiresistive material is wired parallel to the integrated circuit the device is referred to as a p-CARD (Ref. 37). See also U.S. Pat. No. 11,200,474, which is incorporated by reference in its entirety. The pPy coated films were incorporated as the chemiresistors to create p-CARD devices (FIGS. 12A and 12B) capable of measuring glucose concentration using wireless communication. The resonance (reflection coefficient)-frequency trace of the lateral flow-based p-CARD was first measured without the addition of glucose solution using a vector network analyzer and a home-made loop antenna. Solutions with 0, 0.01, 0.05, 0.1, 0.2, 0.3 wt % of glucose together with 0.16 wt % catalyst were added to the pads of different LFA devices and held for 30 minutes wherein all of the solution had left the pad and migrated along the test strip. Resonance-frequency traces of the treated p-CARDs were measured the (FIG. 12C) and a plot of the magnitude of response (ΔGain) versus glucose concentration is shown in (FIG. 12D). An increase in the magnitude of response was observed when the concentration of the glucose solution was at 0.01 wt %. The magnitude of response (ΔGain) increased significantly to 4.3 dB after the addition of 0.3 wt % of glucose. The trend in FIG. 12D demonstrates that our lateral flow device is very sensitive and is capable of accurate detection of glucose concentrations at 0.01 wt % or higher. These results also agree with expectations based on four-point probe and two-point probe conductance measurements. Tests were also conducted for the control lines coated with pyruvate oxidase-conjugated pPy particles and the magnitude of response (ΔGain) also was well-behaved (FIG. 12E and FIGS. 13 and 14). In certain embodiments, the control lines need not be functionalized. In some embodiments, it may only be necessary to confirm that the aqueous fluid has reached the control line and the change in resistivity as a result of the water can be used as a control signal.

In summary, the design and fabrication of a wireless LFA device is described herein that could be used for the detection of an oxidase enzyme response. Methods for creating conducting polymer-enzyme films using bioconjugated pPy core-shell particles were developed that conformally coat nitrocellulose test strips with the polymer in an optimal undoped state. These methods are general and can be used to immobilize other biomolecular elements. The designed pPy core-shell particles exhibit a significant enhancement of conductivity when exposed to H₂O₂ in the presence of a catalyst. Conductivity measurements and resonant radio frequency measurements confirm that this pPy based LFA is able to quantify glucose concentrations in test solutions. The wireless detection with the p-CARD is capable of detecting glucose at or above 0.01 wt %. This device provides new mechanisms for operation of LFAs that can be used in laboratories or for in home testing. LFAs can be created for a variety of assays of interest for healthcare.

### Materials

Unless stated otherwise, all reagents and solvents were used as received without further purification. Glucose Oxidase from Aspergillus niger, Pyruvate Oxidase from microorganisms, (+)-Biotin N-hydroxysuccinimide ester, pyrrole, 1,2-dichlorobenzene (oDCB), sulfolane, Cetrimonium bromide (CTAB), phosphomolybdic acid (PMA), Magnesium sulfate, triethylamine, 3-mercaptopropionic acid were purchased from Sigma-Aldrich. N-Hydroxysuccinimide was purchased from Alfa Aesar. Flavin adenine dinucleotide (FAD) and Thiamine pyrophosphate were purchased from Acros Organics. 1-Ethyl-3-(3-dimethylaminopropyl) carbodiimide hydrochloride (EDC) was purchased from Fluka. AFDye 594 biotin was purchased from Click Chemistry Tools. The phosphovanadomolybdate catalyst (H₅[PV₂Mo₁₀O₄₀]) was prepared according to literature procedures (see, for example, Tsigdinos, G. A.; Hallada, C. J. Molybdovanadophosphoric acids and their salts. I. Investigation of methods of preparation and characterization. Inorg. Chem. 1968, 7, 437-441, which is incorporated by reference in its entirety).

All near field communication (NFC) tags were HF-I Tag-It 13.56 MHz RFID transponder square in-lays made by Texas Instruments and purchased from DigiKey. Nitrocellulose membrane was purchased from GE Healthcare Life Sciences. The 0.020″ thick Backing Cards and 22 mm×300 mm Sample Pad were purchased from DCN Diagnostics.

### Instruments

The optical images of pPy core-shell particles were obtained by an AmScope Trinocular Inverted Microscope equipped with an 18 MP USB 3.0 camera. SEM images of the film and particles were obtained by a Merlin and Crossbeam 540 Zeiss scanning electron microscopy. Four-point probe and two-point probe measurements were conducted by a Keithley 2400 and Signatone Four Point Resistivity System. Thickness of the pPy film on the nitrocellulose membrane was obtained using a Dektak 6M stylus profilometer. Fluorescent microscope images were obtained by a ZEISS AXIO Observer equipped with a ZEISS Axiocam 702 mono Megapixel Microscope Camera. Resonance-frequency traces of the p-CARDs were recorded with an Agilent E5061B 5 Hz-3 GHz Network Analyzer.

The colloidal methods for the synthesis of semiconducting polymers offers an inexpensive scalable method by which materials can be produced for coating a variety of surfaces. There are many applications beyond the formation of test lines in lateral flow assays. Coatings can be used for antistatic applications, printed electronics, stabilization of metal surfaces, coating of textiles, and the depositing of chemiresistive sensors.

The ability of the colloidal platform to allow for the controlled functionalization for polymerized materials is also important. The synthesis of functional monomers is expensive and the polymerizations may not be as efficient for functionalized materials. Functionalization of the as formed colloids is cost effective and the fluidity of the interface allows for facile and homogenous functionalization of the semiconducting materials. Many types of functional groups can be imagined. Groups that provide adhesion to other materials can be included. The functional groups can be used to provide different surface properties of the deposited semiconductor films. In this way films can be made, for example to be hydrophobic or hydrophilic. The materials can be made to bind toxic metal ions such as mercury or lead to create specific interactions with the semiconducting materials for selective detection of these ions. Alternatively other ions such as arsenate can be targeted. Lateral flow assays or related devices for the detection of toxic components in water can be developed. Fluorocarbon functionalization can also be accomplished that will promote the binding of fluorocarbons in water samples. For example, perfluoro-octanoic acide can be detected in lateral flow assays using semiconductors functionalized to have fluorophilic character. Other inorganic ions can be detected by selective binding to receptors, ligands, or through hydrogen bonding. Small molecular organic molecules could may also be selective bound to functionalized semiconducting materials to create lateral flow assays.

### Experimental Procedures

### Preparation of PPy Core-Shell Particles.

The synthesis approaches involved first mix pyrrole, 1,2-dichlorobenzene (oDCB) and sulfolane together as dispersed phase. Then, the aqueous solution containing 0.1 wt % Cetrimonium bromide (CTAB) was added into the dispersed organic phase with volume ratio of organic phase: aqueous phase equals to 1:6. In addition, phosphomolybdic acid (PMA) was introduced into the solution mixture to polymerize pyrrole. After PMA was introduced, a weak reducing agent: sodium sulfite powders were also dispersed into the solution to quench the excess amount of PMA in order to prevent the pyrrole from being doped into a highly-oxidized state. Then the whole mixture was emulsified for 10 seconds to form the stable pPy core shell particles.

### Post-Functionalization Method to Incorporate Carboxyl Groups

The pPy particles were treated with triethylamine to make sure they were completely deprotonated, then followed by thiol addition with 3-mercaptopropionic acid to introduce carboxyl groups. FIGS. 5B-5E illustrated the SEM images of the same amount of pyrrole but different volume ratios of 3-mercaptopropionic acid while FIG. 5A showed SEM image of pPy core-shell particles with no post-functionalization. It can be concluded from FIGS. 5A-5E that with the least amount of 3-mercaptopropionic acid, the pPy particles almost remained a stable shell structure compared with what was shown in FIG. 5A. However, when it reached to the maximum amount of carboxylate contents shown in FIG. 5E, pPy behaved completely bursted onto the SEM silicon wafer substrate.

### Bioconjugation on the PPy Core-Shell Particles.

The carboxyl groups on the particles were activated with the formation of NHS esters. 2 eq. of 1-Ethyl-3-(3-dimethylaminopropyl) carbodiimide hydrochloride and 2 eq. of N-hydroxysuccinimide were added into the continuous phase of pPy particles and reacted for overnight. Then the pPy particles were washed with D.I. water for three times. Streptavidin was connected to the pPy particles by adding 1 eq. of streptavidin into the continuous phase and reacted for 2 hours.

The particles were washed three times with D.I. water to remove the un-reacted streptavidin.

Biotin was chemically connected to glucose oxidase and pyruvate oxidase. We mixed 10 mg of glucose oxidase or pyruvate oxidase with 5 mg of biotin N-hydroxysuccinimide ester in 1 mL of PBS buffer and reacted for overnight. The resulted mixtures were purified by desalting columns. 100 μL of the biotin-glucose oxidase or biotin-pyruvate oxidase solution was added to the continuous phase of the pPy particles (100 μL particles in 1 mL of continuous phase) conjugated with streptavidin for overnight. After washing the particles with D. I. water for three times, the glucose oxidase or pyruvate oxidase functionalized pPy particles were obtained.

### Preparation of PPy Core-Shell Particles Coated Lateral Flow Device.

100 μL of pPy core-shell particles bioconjugated with glucose oxidase and 100 μL of pPy core-shell particles bioconjugated with pyruvate oxidase were added to the nitrocellulose membrane and dried for overnight. The emulsions would be automatically coated onto the membrane. When the films were dried, 0.01 mM Flavin adenine dinucleotide (FAD), 10 mM Magnesium sulfate and 0.2 mM Thiamine pyrophosphate, which were the cofactors, were added to the film coated by pPy particles bioconjugated with pyruvate oxidase. Until the films became dry, then the nitrocellulose membrane was attached to the backing card by attaching the plastic backing of the nitrocellulose to the self-adhesive on the card. The sample pad and the absorbent pad were cut to size and were added to the backing card overlapping with the nitrocellulose membrane by 1 mm.

### Preparation of Poly(Ethylene Dioxythiophene) Core Shell Particles

The formation PEDOT core shell particles, begings with thoroughly mixing the surfactant (polyvinyl alcohol) and reactants (FeCl₃) for half an hour. 3,4-ethylenedioxythiophene was added into the reaction system with mixing and reacting under the surfactant condition for 6 hours at room temperature. After that, the temperature was increased to 90° C. and reacted for 30 min. Finally, the whole system was stirred and reacted at room temperature for another 30 min to one hour. This reaction process produces micron-sized core-shell emulsion particles with diameters of 20 to 40 μm. In this case the core is the ethylene dioxythiophene monomer. With the addition of polystyrene sulfonate into system, slightly larger sized PEDOT core-shell particles will be formed.

### Preparation of Polyaniline Core Shell Particles

Polyaniline, PANI, is polymerized by ammonium persulfate (APS) treatment of a freshly distilled aniline, ortho-dichlorobenzene, sulfolane mixture with 1:1:1 ratio as oil phase and CTAB water solution mixed with 0.1 wt % reduced PMA as continuous phase and surfactants. Introduction of APS and vortexing for 10 times (30 seconds for each time) produced stable PANI core shell emulsions. Thiol addition reactions on these core shell particles increases emulsion stability. PANI core shell particles are obtained conditions wherein thiolates are generated and the addition with 4.5 molar equiv. amounts of the 3-mercaptopropionic acid. These particles can be used for coatings or biofunctionalization.

### Calculation of Conductivity

The conductivity was calculated by using the four-point probe measured resistance values and the thickness values of the coated pPy film. One of the measurements is given below as an example:

D=11 mm;

Before:

ρₛ=R*C=4.2548*138.0484 MΩ*1000=587368.332

Bulk Resistivity: 569903.321 Å*587368.332*10⁻¹⁰=33.474

Conductivity=1/Bulk Resistivity=2.987*10⁻⁴ S/cm

After:

ρₛ=R*C=4.2548*18.668 KΩ=79.429

Bulk Resistivity: 569903.321 Å*79.429*10⁻¹⁰=4.527*10⁻³ S/cm

Conductivity=1/Bulk Resistivity=2.209 S/cm

### RFID Measurement

The sample solutions were added to the sample pad of the lateral flow device for 30 mins. And when the films were dry, the vector network analyzer and a loop were used to record the resonance (reflection coefficient, or S11 parameter)-frequency traces. The probe was placed above the lateral flow device at a consistent distance to keep all the parameters consistent.

### REFERENCES

Each of the following references is incorporated by reference in its entirety.
(1) Napoli, J. L., Biochemical Pathways of Retinoid Transport, Metabolism, and Signal Transduction. J. Clin. Immunol. 1996, 80, S52-S62.
(2) Muñoz, B.; Suárez-Sánchez, R.; Hernández-Hernández, O.; Franco-Cendejas, R.; Cortés, H.; Magaña, J. J., From Traditional Biochemical Signals to Molecular Markers for Detection of Sepsis After Burn Injuries. Burns 2019, 45, 16-31.
(3) Banes, A. J.; Tsuzaki, M.; Yamamoto, J.; Brigman, B.; Fischer, T.; Brown, T.; Miller, L., Mechanoreception at the Cellular Level: the Detection, Interpretation, and Diversity of Responses to Mechanical Signals. Biochem. Cell Biol. 1995, 73, 349-365.
(4) Chen, W. W.; Niepel, M.; Sorger, P. K., Classic and Contemporary Approaches to Modeling Biochemical Reactions. Genes Dev. 2010, 24, 1861-1875.
(5) Zhang, Y.; Narlikar, G. J.; Kutateladze, T. G., Enzymatic Reactions inside Biological Condensates. J. Mol. Biol. 2021, 433, 166624.
(6) Ryabov, A. D., The Biochemical Reactions of Organometallics with Enzymes and Proteins. Angew. Chem. Int. Ed. 1991, 30, 931-941.
(7) Beilen, J. B. v.; Li, Z., Enzyme Technology: An Overview. Curr. Opin. Biotechnol. 2002, 13, 338-344.
(8) R. M. Daniel; R. V. Dunn; J. L. Finney; Smith, J. C., The Role of Dynamics in Enzyme Activity. Annu. rev. biophys. biomol. struct. 2003, 32, 69-92.
(9) Hunt, J. F.; Earnest, T. N.; Bousché, O.; Kalghatgi, K.; Reilly, K.; Horváth, C.; Rothschild, K. J.; Engelman, D. M., A Biophysical Study of Integral Membrane Protein Folding. Biochemistry 1997, 36, 15156-15176.
(10) Gerlt, J. A.; Allen, K. N.; Almo, S. C.; Armstrong, R. N.; Babbitt, P. C.; Cronan, J. E.; Dunaway-Mariano, D.; Imker, H. J.; Jacobson, M. P.; Minor, W.; Poulter, C. D.; Raushel, F. M.; Sali, A.; Shoichet, B. K.; Sweedler, J. V., The Enzyme Function Initiative. Biochemistry 2011, 50, 9950-9962.
(11) Ringe, D.; Petsko, G. A., Biochemistry. How Enzymes Work. Science 2008, 320, 1428-1429.
(12) Wong, C. M.; Wong, K. H.; Chen, X. D., Glucose Oxidase: Natural Occurrence, Function, Properties and Industrial Applications. Appl. Microbiol. Biotechnol. 2008, 78, 927-938.
(13) Fu, L.-H.; Qi, C.; Lin, J.; Huang, P., Catalytic Chemistry of Glucose Oxidase in Cancer Diagnosis and Treatment. Chem. Soc. Rev. 2018, 47, 6454-6472.
(14) Bankar, S. B.; Bule, M. V.; Singhal, R. S.; Ananthanarayan, L., Glucose oxidase—An overview. Biotechnology Advances 2009, 27, 489-501.
(15) Mano, N., Engineering Glucose Oxidase for Bioelectrochemical Applications. Bioelectrochemistry 2019, 128, 218-240.
(16) Raba, J.; Mottola, H. A., Glucose Oxidase as an Analytical Reagent. Crit. Rev. Anal. Chem. 1995, 25, 1-42.
(17) Bankar, S. B.; Bule, M. V.; Singhal, R. S.; Ananthanarayan, L., Glucose Oxidase—An Overview. Biotechnol. Adv. 2009, 27, 489-501.
(18) Bateman, R. C.; Evans, J. A., Using the Glucose Oxidase/Peroxidase System in Enzyme Kinetics. J. Chem. Educ. 1995, 72, A240.
(19) Yuen, V. G.; McNeill, J. H., Comparison of the Glucose Oxidase Method for Glucose Determination By Manual Assay and Automated Analyzer. J Pharmacol. Toxicol. Methods. 2000, 44, 543-546.
(20) Sung, W. J.; Bae, Y. H., A Glucose Oxidase Electrode Based on Polypyrrole with Polyanion/PEG/Enzyme Conjugate Dopant. Biosens. Bioelectron. 2003, 18, 1231-1239.
(21) Yusan, S.; Rahman, M. M.; Mohamad, N.; Arrif, T. M.; Latif, A. Z. A.; M. A, M. A.; Wan Nik, W. S. B., Development of an Amperometric Glucose Biosensor Based on the Immobilization of Glucose Oxidase on the Se-MCM-41 Mesoporous Composite. J. anal. methods chem. 2018, 2018, 2687341.
(22) Wilson, R.; Turner, A. P. F., Glucose Oxidase: An Ideal Enzyme. Biosens. Bioelectron. 1992, 7, 165-185.
(23) Sajid, M.; Kawde, A.-N.; Daud, M., Designs, Formats and Applications of Lateral Flow Assay: A Literature Review. J. Saudi Chem. Soc. 2015, 19, 689-705.
(24) Koczula, Katarzyna M.; Gallotta, A., Lateral Flow Assays. Essays Biochem. 2016, 60, 111-120.
(25) Raeisossadati, M. J.; Danesh, N. M.; Borna, F.; Gholamzad, M.; Ramezani, M.; Abnous, K.; Taghdisi, S. M., Lateral Flow Based Immunobiosensors for Detection of Food Contaminants. Biosens. Bioelectron. 2016, 86, 235-246.
(26) Jiang, N.; Ahmed, R.; Damayantharan, M.; Ünal, B.; Butt, H.; Yetisen, A. K., Lateral and Vertical Flow Assays for Point-of-Care Diagnostics. Adv. Healthc. Mater. 2019, 8, 1900244.
(27) Bahadir, E. B.; Sezgintürk, M. K., Lateral Flow Assays: Principles, Designs and Labels. Trends Analyt. Chem. 2016, 82, 286-306.
(28) Posthuma-Trumpie, G. A.; Korf, J.; van Amerongen, A., Lateral Flow (Immuno)Assay: Its Strengths, Weaknesses, Opportunities and Threats. A Literature Survey. Anal. Bioanal. Chem. 2009, 393, 569-582.
(29) Smits, H. L.; Eapen, C. K.; Sugathan, S.; Kuriakose, M.; Gasem, M. H.; Yersin, C.; Sasaki, D.; Pujianto, B.; Vestering, M.; Abdoel, T. H.; Gussenhoven, G. C., Lateral-Flow Assay for Rapid Serodiagnosis of Human Leptospirosis. Clin. Vaccine Immunol. 2001, 8, 166-169.
(30) Goodrum, P. M.; McLaren, M. A.; Durfee, A., The Application of Active Radio Frequency Identification Technology for Tool Tracking on Construction Job Sites. Autom. Constr. 2006, 15, 292-302.
(31) Li, S.; Visich, J. K.; Khumawala, B. M.; Zhang, C., Radio Frequency Identification Technology: Applications, Technical Challenges and Strategies. Sens. Rev. 2006, 26, 193-202.
(32) Ashar, B. S.; Ferriter, A., Radiofrequency Identification Technology in Health CareBenefits and Potential Risks. JAMA 2007, 298, 2305-2307.
(33) Petersen, C. L.; Hansen, T. M.; Bøggild, P.; Boisen, A.; Hansen, O.; Hassenkam, T.; Grey, F., Scanning Microscopic Four-Point Conductivity Probes. Sens. Actuator A Phys. 2002, 96, 53-58.
(34) Petersen, D. H.; Hansen, O.; Hansen, T. M.; Bøggild, P.; Lin, R.; Kjær, D.; Nielsen, P. F.; Clarysse, T.; Vandervorst, W.; Rosseel, E.; Bennett, N. S.; Cowern, N. E. B., Review of Electrical Characterization of Ultra-Shallow Junctions with Micro Four-Point Probes. J. Vac. Sci. Technol. B 2010, 28, C1C27-C1C33.
(35) Crocker, J. C.; Valentine, M. T.; Weeks, E. R.; Gisler, T.; Kaplan, P. D.; Yodh, A. G.; Weitz, D. A., Two-Point Microrheology of Inhomogeneous Soft Materials. Phys. Rev. Lett. 2000, 85, 888-891.
(36) Want, R., Near Field Communication. IEEE Pervasive Comput. 2011, 10, 4-7.
(37) Zhu, R.; Azzarelli, J. M.; Swager, T. M., Wireless Hazard Badges to Detect Nerve-Agent Simulants. Angew. Chem. Int. Ed. 2016, 55, 9662-9666.
(38) Coskun, V.; Ozdenizci, B.; Ok, K., A Survey on Near Field Communication (NFC) Technology. Wirel. Pers. Commun. 2013, 71, 2259-2294.
(39) Allah, M. M. A. J. G. j. o. c. s.; technology, Strengths and Weaknesses of Near Field Communication (NFC) Technology. J. Comput. Sci. Technol. 2011.
(40) Li, H.; Han, D.; Hegener, M. A.; Pauletti, G. M.; Steckl, A. J., Flow Reproducibility of Whole Blood and Other Bodily Fluids in Simplified No Reaction Lateral Flow Assay (nrLFA) Devices. Biomicrofluidics 2017, 11, 024116.
(41) Tiwari, C. K.; Baranov, M.; Neyman, A.; Neumann, R.; Weinstock, I. A., Selective Oxidation by H5[PV2Mo10O40] in a Highly Acidic Medium. Inorg. Chem. 2020, 59, 11945-11952.
(42) Leng, Y.; Ge, H.; Zhou, C.; Wang, J., Direct Hydroxylation of Benzene with Hydrogen Peroxide Over Pyridine-Heteropoly Compounds. Chem. Eng. J. 2008, 145, 335-339.

Details of one or more embodiments are set forth in the accompanying drawings and description. Other features, objects, and advantages will be apparent from the description, drawings, and claims. Although a number of embodiments of the invention have been described, it will be understood that various modifications may be made without departing from the spirit and scope of the invention. It should also be understood that the appended drawings are not necessarily to scale, presenting a somewhat simplified representation of various features and basic principles of the invention.

1. A device for detecting an analyte comprising:
a porous substrate capable of transporting a sample through capillary action; and
a sensing region including a semiconducting material on a surface of the porous substrate and a recognition moiety configured to respond to the analyte;
wherein the sensing region has an electronic property that changes in response to the analyte.

2. The device of claim 1, wherein the semiconducting material includes a semiconducting polymer film, a carbon nanotube, or an MXene film on the surface of the porous substrate.

3. The device of claim 2, wherein the semiconducting polymer film includes a polypyrrole, a polythiophene, a polyaniline, a polyacetylene, a polyphenylene, a polyarylene, a polyphenylene vinylene, or polyarylene vinylene.

4. The device of claim 1, wherein the semiconducting material is initially in a lower conductive state than that after analyte detection.

5. The device of claim 1, wherein the semiconducting material is initially in a higher conductive state than that after analyte detection.

6. The device of claim 1, wherein the analyte detection results in a change in conductivity of at least 10%.

7. The device of claim 1, wherein the analyte detection results in a change in conductivity of about 100%.

8. The device of claim 1, wherein the recognition moiety includes an enzyme, protein, synthetic receptor, antibody, nano-body, nucleic acid, molecular catalyst, metal binding site, or combinations thereof.

9. The device of claim 8, further comprising a redox catalyst adjacent to the semiconducting material.

10. The device of claim 8, further comprising a redox catalyst initially in an analyte solution capable of reacting with the semiconducting material.

11. The device of claim 1, wherein the sensing region includes an enzyme.

12. The device of claim 1, wherein the analyte includes a bacterium, a protein, a virus, a nucleic acid, a cell, a reactive biomarker, an enzyme substrate, a carbohydrate, a highly fluorinated molecule, a toxic molecule, a metal ion, or combinations thereof.

13. The device of claim 1, wherein the analyte is capable of binding to or reacting with an enzyme or capable of binding to two or more recognition moieties at the same time.

14. The device of claim 1, wherein the porous substrate further includes a control region adjacent to the sensing region.

15. The device of claim 14, wherein the porous substrate includes a sampling region, the sampling region being adjacent to the control region or the sensing region.

16. The device of claim 1, further comprising a catalyst.

17. The device of claim 1, wherein the electronic property is resistivity.

18. The device of claim 1, further comprising a radio frequency identification tag electrically connected to the sensing region.

19. The device of claim 18, wherein the radio frequency identification tag includes an integrated circuit in parallel with the sensing region.

20. The device of claim 18, wherein the radio frequency identification tag includes an integrated circuit in series with the sensing region.

21. A system for detecting an analyte comprising:
the device of claim 1, and
a reader configured to measure the electronic property of the sensing region.

22. The system of claim 21, wherein the reader includes a wireless reader.

23. The system of claim 21, wherein the wireless reader is a smartphone.

24-49. (canceled)
